(19) United States
(12) Patent Application Publication
(10) Pub. No.: US 2006/0276776 A1
(43) Pub. Date: Dec. 7, 2006

(54) Method and system for two-step customized cornea reshaping using ultraviolet infrared lasers

(76) Inventor: J. T. Lin, (US)

Correspondence Address: J. T. Lin, 4532 Old Carriage Trail, Oviedo, FL, 32765, US

(21) Appl. No.: 11/140,905

(22) Filed: Jun. 1, 2005

(51) Int. Cl. A61B 18/18

(52) U.S. Cl. 606/5

(57) ABSTRACT

Method and systems for customized two-step corneal reshaping using an elevation map and adjustable laser spot size are disclosed. System includes lasing crystals (Nd:YAG, Nd:YLF, Nd:YVO4, Er:YAG, Er:Cr:YSGG and Er:YSGG), nonlinear crystals (KTP, KDP, CDA, CBO, BBO and LBO), scanning optics and beam spot control means. Flash-lamp or diode-laser pumped lasers with output wavelength at UV (193 to 266 nm) and mid-IR (2.7 to 2.94 microns) are preferred. The customized ablation profile may be calibrated by PMMA based on the pre-operation profile and the calculated profiles for emmetropia.

### BACKGROUND OF THE INVENTION

1. Field of the Invention

This invention relates to method and system for refractive surgery using laser to ablate the corneal surface. It also relates to system design for customized ablation for irregular corneal surface.

2. Prior Art

Two major technologies have been developed from cornea surface reshaping for vision corrections: the “broad beam” patented by L'Esperance in U.S. Pat. Nos. 4,580,559; 4,665,913 and the “flying spot” scanning beam system patented by the present inventor in U.S. Pat. Nos. 5,144,630; 5,520,679 (referred as Lin-630 and Lin-679). Development of advanced corneal mapping device such as Obscan, provides the localized elevation map (EM) of the corneal front surface. The EM provides more information than the conventional map (CM) which is based on “averaged curvature” to define an overall diopter of the corneal surface. For irregular corneal surface or off-centered cornea after the refractive surgery, corneal diopter is no longer well defined and localized (or customized) information based on an EM is required in vision correction. Recent technology of wave-front device further provides the optical aberration (OA) measurement of the eye such that so called “super-vision” (better than 20/20) becomes possible by eliminating the OA.

Existing system using ArF excimer laser for a procedure called laser in situ keratomileusis (LASIK) for customized corneal reshaping (CCR) is based on average corneal surface data or CM, therefore, only “approximate” large area correction is available. A true, localized correction based on EM has not yet been developed. CCR based on CM has other drawbacks. For example, in correcting an off-centered eye (after refractive surgery), corneal surface ablation is conducted by removing a portion of the off-centered area and then make another correction on the resultant refractive error, where “symmetry” ablation profiles are used and the power correction is defined by an averaged diopter. This procedure most of the time will ablate too much corneal tissue with the high risk of corneal weakening, and can only correct symmetric-type errors. Existing system input parameters based on surface diopter and CM, therefore, can not correct localized irregularity of the surface which requires the EM data and the calculated profile difference, defined by the difference between desired profile and the initial profile.

In addition, all the prior arts are using a fixed laser beam spot size which limits the accuracy of corrected profile, particularly when the localized irregularity is smaller than the beam spot, typically about 0.8 to 1.5 mm in existing systems for LASIK. We also note that smaller beam spot provides more accurate ablation profile but slower procedure, whereas larger spot gives faster procedure with poor accuracy. Prior arts such as Lin-679 or Lin-630 did not optimize these two competing factors. There is no system available for LASIK procedures using adjustable beam spot size which is one of the critical elements of CCR based on the teaching of the present invention.

One objective of the present invention is to use a scanning beam system and an EM for a true localized (customized) correction without using CM or the averaged diopter.

Yet another objective of the present invention is to define the procedures (or steps) allowing the surgeon to test the desired profile on PMMA (a plastic sheet) prior to the surgery on cornea.

Yet another objective of the present invention includes examples of irregular corrections based on the calculated profile difference defined earlier.

Yet another objective of the present invention includes the control means of laser beam spot size for optimal clinical outcomes including accuracy of the correcting profile and fast surgery procedure.

### SUMMARY OF THE INVENTION

The present invention discloses a scanning beam system consisting of a pair of scanner, a beam spot control means and light source. EM for a true localized (customized) correction without using CM or the averaged diopter is proposed to define the corneal surface profile.

One preferred embodiment of this invention includes procedures (or steps) allowing the surgeon to test the desired profile on PMMA (a plastic sheet) prior to the surgery on cornea.

Yet another preferred embodiment of the present invention includes customized profiles for the correction of off-centered surface, irregular myopia, hyperopia or astigmatism.

Yet another preferred embodiment of the present invention includes the control means of laser beam spot size using focusing lens combination, iris or shutter for adjustable spot size of about 0.2 to 3.0 mm.

Yet another preferred embodiment of the present invention includes the use of overlapped Gaussian beam for smooth cornea surface after the surgery.

Yet another preferred embodiment of the present invention includes a laser having a wavelength of about 193 to 266 nm and 2.7 to 2.94 microns generated from a flash-lamped or diode-laser pumped system.

Further preferred embodiments of the present invention will become apparent from the detailed description of the invention which follows.

### BRIEF DESCRIPTION OF THE DRAWINGS

FIG. 1. Schematics of the system for customized cornea reshaping (CCR).

FIG. 2. Schematics of a laser beam spot control means.

FIG. 3. System parameters and testing flow chart of CCR.

FIG. 4. Examples of irregular corrections and the profile difference Between the desired and initial profile.

### DETAILED DESCRIPTION OF THE INVENTION AND THE PREFERRED EMBODIMENTS

FIG. 1 shows the schematics of the system which consists of a microprocessor 1 connected to the laser unit 2 having an output beam 3 which is passing through a beam spot control means 4. A pair of X-Y motion scanning optics 5 is used to control the ablation pattern on the treated cornea surface 6 which can be viewed by a microscope via another 45 degree angle reflecting optics 8. The preferred embodiment may also include a visible aiming beam collinear to the laser output 3 and an eye tracker (not shown in FIG. 1) to follow eye motion during the surgery. The basic laser in FIG. 1 shall include ArF laser at 193 nm, the fourth or fifth harmonics of Nd:YAG, Nd:YLF or Nd:YVO4 at 212 to 266 nm, and Er:YAG, Er:YSGG, Er:Cr:YSGG, Er:YALO3 at about 2.7 to 2.94 microns, having a pulse duration of about 1.0 nanosecond to 700 microsecond, repetition rate of about 5 to 500 Hz.

The solid-state lasers can be flash-lamp pumped or diode-laser pumped, where the diode-laser includes a pumping wavelength about 810 nm (for UV output) or about 750 to 980 nm (for IR output) and power of about 5 to 50 W. The preferred energy of the laser on the corneal surface includes about 0.5 to 10 mJ for UV laser (at about 193 to 266 nm) or about 5 to 50 mJ for IR laser (at about 2.7 to 2.94 micron), having a spot size (on corneal surface) of about 0.2 to 3.0 mm. The solid-state UV laser at about 212 to 266 nm is generated from the use of nonlinear crystals including KTP, KDP, CDA, CBO, BBO or LBO.

For the case of diode-laser-pumped UV or IR laser, the preferred laser cavity configuration is side-pumping, where the pumping diode laser array is located at both sides of the laser crystal. The most preferable flash-lamped or diode-pumped UV laser is about 264 to 266 nm generated from the fourth-harmonic of Nd:YAG or Nd:YLF, which is much easier to obtain the required UV energy (about 3 to 8 mJ on corneal surface) than the fifth-harmonic at about 213 nm, proposed by prior arts of Lin-630. We also note that side-pumping configuration is required to generate sufficient energy required for LASIK procedure. In comparison, the end-pumping configuration (having the diode-laser located at one end of the laser crystal) has much lower pumping efficiency is excluded in the present invention. The above disclosed configuration and parameters, which are critical in diode-pumped systems, have not been disclosed in prior arts.

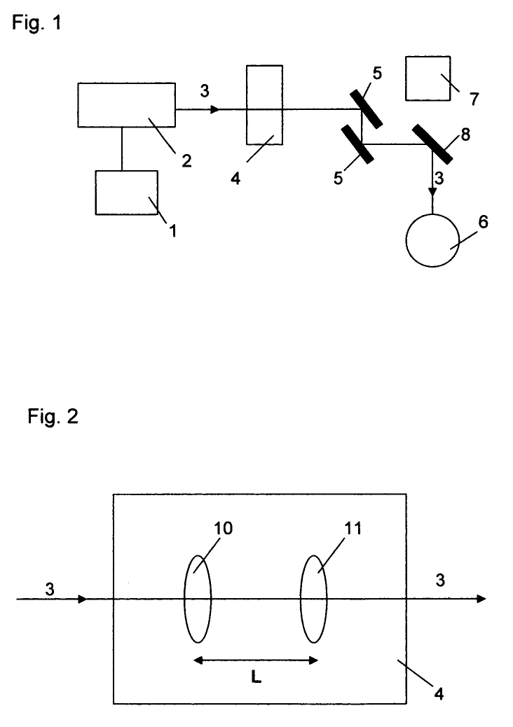

FIG. 2 shows the preferred structure of the beam spot control means 4 which includes a pair of focusing lens (could be a combination of spherical or cylinder lens) 10 and 11 having a focal length of F1 and F2, with about F1=(20 to 200) cm and F2=(100 to 500) cm. By adjusting F1 and F2 and their separation L about (1.0 to 10) cm, we may obtain the preferred laser beam spot size (on the corneal surface) about 0.2 to 3.0 mm. The preferred embodiment of the beam spot control means shall also include electrical shutter, iris, pin-hole or the combination with focusing lenses. One preferred combining focal length F1+F2 shall be about the distance from the first lens 10 to the cornea surface 6. The output beam 3 after the control means 4 is preferred to be slightly focused and most preferable to be collimated.

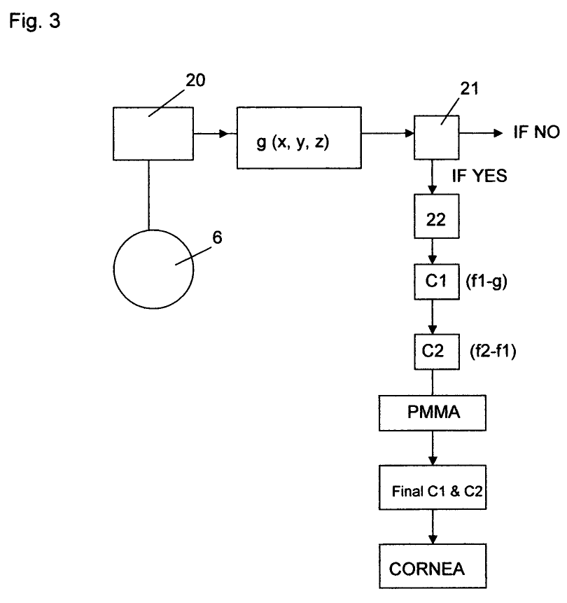

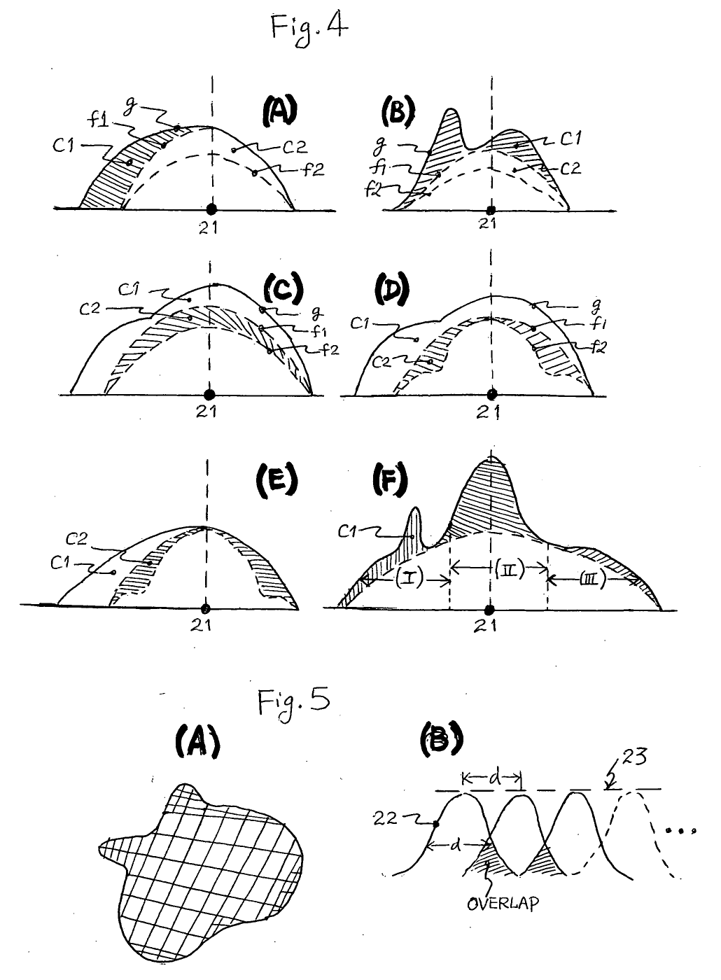

As shown by the chart of FIG. 3 g(x,y,z) is the initial profile of the corneal surface measured by an elevation-map (EM) device 20, such as the commercial one made by Obscan. The decision step 21 is made to decide if CCR (customized) or a regular correction will be needed based on the EM of g, such as asymmetry, off center, or irregular local spots. The calculation step 22 further consists of two steps: (1) find the first correcting profile C1 given by C1=f1(x,y,z)−g(x,y,z), which corrects the irregularity or off-center of the initial profile g, f1 being the profile of a regular, symmetric profile; and (2)use the second correcting profile C2=f2−f1 to achieve the final desired profile including the desired correction power for emmetropia. The final profile may be tested by ablating a PMMA (a plastic sheet) as follows. First, one needs to generate the initial profile g on the flat surface of PMMA, then use the calculated C1=f1−g to generate the profile f1 which shall be checked if it represents symmetric profile with respect to the center point. The desired profile f2 may be generated on PMMA by ablation a myopia or hyperopia correction profile given by the calculated C2. For example, if f1 shows a myopia error of D, then f2 shall have a profile of
h(r,z)=0.33D(W−r)²−C,
where r is the radial coordination of the cornea and W is the ablation zone diameter (in mm), and the profile depth (h) in micron. We had added a new correction term C=0.06W⁴/R², with R being the cornea anterior curvature relating to the keratometry reading by R=377/K. This new formula, more accurate than existing one, was recently published by the present inventor (Lin, in J. Refract. Surgery, vol. 21, 200-201, 2005). The new formulas include the effect of cornea curvature and require addition pre-operation K-reading which was ignored in prior arts.

Fine adjustment may be made after measuring the f2 profile on PMMA, including the ablation power which needs to be adjusted to human corneal tissue by a factor of about 3, that is, the tissue ablation rate is about 3 times more than PMMA, or one diopter on PMMA equivalent to about 3 diopters on cornea surface. The finalized C1 and C2 then can be used on patient.

There are several technical aspects which are critical for CCR. The profiles of g, f1 and f2 are all x,y,z dependent. The existing system used a symmetric profile assuming no difference in x and y, which are not assumed in the present invention and the EM data is used to define the z-dependence of both x and y. To obtain a smooth corneal surface after the laser ablation, about 50% to 70% of beam spot overlap Gaussian profile (rather than flat-top) are preferred. To achieve the accuracy of correction profiles C1 and C2, the laser beam spot size must not larger than the local irregular area, which could be about 0.2 to 4.0 mm. Therefore, the preferred beam spot includes about 0.2 to 3.0 mm, and it is adjustable according to the size of the localized initial irregularity of the corneal surface. Greater detail will be shown in the following examples of off-centered surface, asymmetric astigmatism, irregular myopia, irregular hyperopia correction and other irregular surface.

### EXAMPLE 1

### Off-Center Correction

As shown in FIG. 4(A), the initial corneal surface profile (g) is off-centered (with cornea center at 21) after a refractive surgery and needs a re-treatment. The first step is to remove the surface tissue based on the area defined by C1=f1−g, which corrects the off-center, then followed by a second-step of C2=f2−f1 to treat any remaining refractive error (if any). This example shows a myopic correction after the profile is centered.

### EXAMPLE 2

### Asymmetric Astigmatism

As shown in FIG. 4(B), C1 defines the area of corneal tissue needed to be removed to eliminate the asymmetric surface, followed by C2 which corrects a remaining myopia in this example. The final profile f2 becomes symmetric and emmetropic.

### EXAMPLE 3

### Irregular Myopia

Shown in FIG. 4(C) which also requires two steps to achieve the piano (or emmetropia) profile f2: the irregular correction step C1, followed by a myopia correction C2. We note that if there is an overcorrection of step C1, then a hyperopia correction in step C2 will be required, as shown in FIG. 4(D).

### EXAMPLE 4

### Irregular Hyperopia

FIG. 4(E) shows step C1 to make the corneal surface symmetric which is followed by a hyperopia correction profile C2.

### EXAMPLE 5

### Localized Irregularity

As shown in FIG. 4(F), there are three different sizes (or degrees) of irregularities which may be smoothened by, for example, a laser spot size of about 0.2 mm to correct the “steep” area (I), 0.6 mm to correct area (II) and a large spot of about 2.0 mm for larger and smoother area (III). System with adjustable spot size allows us to correct all degree of irregularity, whereas the prior arts with a typical spot of 1.0 mm could not accurately correct areas (I) and (II), and it also takes about four times longer in correcting area (III) comparing to that of a larger spot (2.0 mm) proposed in this invention.

We shall also note that drawings of the above-examples are shown in an elevation map just in 2 dimensions (say x-z). The other y-z plane was not shown. The 3-dimensional EM makes the customized LASIK more difficult due to its lack of symmetry in general. As shown in FIG. 5, a “mesh point” is needed to define an irregular shape of corneal surface, where more square units would be needed for areas nearby the boundary. Another important issue of CCR is the roughness after the ablation, particularly when spot size smaller than 0.5 mm is used. Therefore, one of the preferred embodiment is to use a Gaussian profile beam (rather than a flat-top), and have an overlap about 50% to 70% for each scanning spot. As shown in FIG. 5(B), a smooth surface may be achieved easily by a Gaussian profile with overlap, but not in flat-top which would require a perfect matching without overlap. FIG. 5(B) shows a 50% overlap case, where the peak-to-peak of the Gaussian profile 23 approximately equals to its profile width (d). The straight line 23 shows the approximate sum of the Gaussian profiles.

Yet another important issue of CCR is how to convert the EM to the number of laser pulse required in each of the mesh point shown in FIG. 5. For example, the measured ablation depth per pulse (defined as A1) at a given laser energy, equals to the ablation depth after a 50% overlap scanning beam. Therefore, the total depth after N-scanning will be NA1, where typical value of A1 is about 0.2 to 0.5 microns. So, a 5-15 diopter myopia with an effective optical zone of 6.5 mm in a so called 3-16 zone method published the present inventor ( in J. Refract. Surgery, 2005, in press) will require a center depth of about 5×0.79×42/3=55 microns, which will require about 55/0.3=183 pulse (or scanning layers), or about 1.8 seconds for a laser operated at 100 Hz. If a total of 10 scanning layers are needed, the surgical procedure will take about 18 seconds to complete. Above analysis is based on a 1.0 mm spot size. It will be reduced by a factor of 2.25 (or about 8 seconds) when a 1.5 mm spot is used, whereas it will take about 28 seconds when a 0.8 mm spot is used.

Above examples show that a larger spot is preferred for a faster procedure. However, larger spot suffers accuracy on ablation profile, particularly for the correction of small area irregularity. Therefore, another preferred embodiment of this invention is to use various spot size at various stage (or ablation area) of the procedure. For example, spot size R=(0.2 to 0.5) mm is preferred to correct a localized small area regularity, R=(0.8 to 1.2) mm is preferred for ablation of the inner zone of the 3-zone method, and R=(1.5 to 2.5) mm to treat the outer or transition zone. We note that the above optimal ablation for customized or regular procedure governed by three different spot sizes are not possible based on prior arts using one fixed spot size.

In addition to the above discussed irregular patterns, this invention also applies to regular patterns or non-customized LASIK, where the use of adjustable beam spot may shorten the procedure time and may also be used to minimize corneal surface aberration by reshaping the cornea to a “prolate” profile or an aspherical surface away from its vertex.

I claim:

1. A surgical method for customized corneal reshaping, comprising the steps of:
(a) selecting a laser beam having a predetermined energy, spot size and wavelength;
(b) selecting a means of beam spot size control;
(c) selecting a scanning optics which delivers said laser beam energy to a predetermined area with a predetermined ablation pattern of the corneal surface;
(d) selecting a calibration means for the predetermined ablation pattern; and whereby refractive error of the treated eye is corrected based on the measured elevation map.

2. A surgical method of claim 1, wherein said laser beam is an ultraviolet laser having a wavelength 193 nm or 212 to 266 nm and a pulse energy of about 0.5 to 10 mJ, pulse width of about 1.0 to 20 ns on said corneal surface.

3. A surgical method of claim 1, wherein said laser beam having a wavelength about 264 to 266 nm is generated from a flash-lamp or diode-laser pumped Nd:YAG, Nd:YVO4, or Nd:YLF and frequency converted by harmonic generation nonlinear crystals of KTP, KDP, CDA, CBO, BBO or LBO.

4. A surgical method of claim 3, wherein said diode-laser is a semiconductor diode array having a wavelength about 810 nm power of about 5 to 50 W and used in a side-pumping configuration.

5. A surgical method of claim 1, wherein said laser beam is generated from a flash-lamp or diode-laser pumped infrared laser of Er:YAG, Er:YSGG, Er:Cr:YSGG or Er:YALO3 having an output wavelength of about 2.7 to 2.94 microns, energy per pulse about 5 to 50 mJ and pulse width about 100 to 700 microsecond on said corneal surface.

6. A surgical method of claim 5, wherein said diode-laser is a semiconductor diode array having a wavelength about 750 to 980 nm and power of about 5 to 50 W and used in a side-pumping configuration.

7. A surgical method of claim 1, wherein said predetermined ablation pattern is defined by the measured elevation profile pre-operation (g) and the calculated profile for emmetropia (f1 and f2), where said predetermined ablation pattern is governed by two steps correction defined by C1=f1−g and C2=f2−f1.

8. A surgical method of claim 1, wherein said calibration means includes the use of PMMA to test the customized correction profile.

9. A surgical method of claim 1, wherein said means of a beam spot size control includes the use of focusing spherical or cylinder lens having focal length of about 20 to 500 cm, electrical shutter, iris, or pin-hole, whereby said laser beam spot size is adjustable between about 0.2 and 3.0 mm on said corneal surface.

10. A surgical method of claim 9, wherein said laser beam spot size about 0.2 to 1.0 mm is used for the correction of small irregular area, and large spot about 1.0 to 3.0 mm is used for larger irregular area on said corneal surface.

11. A surgical system for customized corneal reshaping, comprising of:
(a) a laser beam having a predetermined energy, spot size and wavelength;
(b) a means of beam spot size control;
(c) a scanning optics which delivers said laser beam energy to a predetermined area with a predetermined ablation pattern of the corneal surface;
(d) a calibration means for the predetermined ablation pattern; and whereby refractive error of the treated eye is corrected based on the measured elevation map.

12. A surgical system of claim 11, wherein said laser beam is an ultraviolet laser having a wavelength 193 nm or 212 to 266 nm and a pulse energy of about 0.5 to 10 mJ, pulse width of about 1.0 to 20 ns on said corneal surface.

13. A surgical system of claim 11, wherein said laser beam having a wavelength about 264 to 266 nm is generated from a flash-lamp or diode-laser pumped Nd:YAG, Nd:YVO4, or Nd:YLF and frequency converted by harmonic generation nonlinear crystals of KTP, KDP, CDA, CBO, BBO or LBO.

14. A surgical system of claim 13, wherein said diode-laser is a semiconductor diode array having a wavelength about 810 nm power of about 5 to 50 W and used in a side-pumping configuration.

15. A surgical system of claim 11, wherein said laser beam is generated from a flash-lamp or diode-laser pumped infrared laser of Er:YAG, Er:YSGG, Er:Cr:YSGG or Er:YALO3 having an output wavelength of about 2.7 to 2.94 microns, energy per pulse about 5 to 50 mJ and pulse width about 100 to 700 microsecond on said corneal surface.

16. A surgical system of claim 15, wherein said diode-laser is a semiconductor diode array having a wavelength about 750 to 980 nm and power of about 5 to 50 W and used in a side-pumping configuration.

17. A surgical method of claim 1, wherein said calibration means includes the use of PMMA to test the customized correction profile.

18. A surgical system of claim 11, wherein said means of a beam spot size control includes the use of focusing spherical or cylinder lens having focal length of about 20 to 500 cm, electrical shutter, iris, or pin-hole, whereby said laser beam spot size is adjustable between about 0.2 and 3.0 mm on said corneal surface.

19. A surgical system of claim 18, wherein said laser beam spot size about 0.2 to 1.0 mm is used for the correction of small irregular area, and large spot about 1.0 to 3.0 mm is used for larger irregular area on said corneal surface.
